(19) United States
(12) Patent Application Publication
(10) Pub. No.: US 2014/0294944 A1
(43) Pub. Date: Oct. 2, 2014

(54) MICROENCAPSULATION OF OXYGEN LIBERATING REACTANTS

(71) Applicant: Kimberly-Clark Worldwide, Inc., Neenah, WI (US)

(72) Inventor: Iris V. Schumacher, Gainesville, FL (US)

(73) Assignee: Kimberly-Clark Worldwide, Inc., Neenah, WI (US)

(21) Appl. No.: 14/205,543

(22) Filed: Mar. 12, 2014

Related U.S. Application Data
(60) Provisional application No. 61/806,075, filed on Mar. 28, 2013.

(51) Int. Cl. A61K 9/50; A61K 47/42; A61K 33/40

(52) U.S. Cl. CPC A61K 9/50; A61K 33/40; A61K 47/42
USPC 424/451; 424/616; 424/59

(57) ABSTRACT

There is provided a composition for the delivery of oxygen having microencapsulated peroxide and microencapsulated catalyst that liberate oxygen upon sufficient contact with each. The components may be stored separately or together until use. Upon mixing, oxygen is liberated. The composition can be used for wound healing or for cosmetic applications to deliver oxygen to the skin to help skin elasticity and retard the effects of aging.

This application claims the benefit of priority from U.S. Provisional Application No. 61/806,075 filed on Mar. 28, 2013, the contents of which are incorporated herein by reference.

The present disclosure relates to the provision of oxygen for use in cosmetic and wound healing formulations.

The lack of oxygen, i.e. hypoxia, is commonly experienced by people in their extremities as they get older due to poor blood circulation as well as by those with conditions such as diabetes. Studies have also shown below normal, low oxygen tension in the skins of older people. This often leads to poor skin health and an excessive presence of visible conditions such as wrinkles, dryness and lower skin elasticity. Over the years, cosmetic manufacturers have introduced skin formulations with a large variety of ingredients such as emollients, exfoliators, moisturizers etc., to retard these age related effects and improve and maintain skin health. Attacking the problem of low oxygen directly has not been generally practiced.

The delivery of oxygen to the skin for common use is a technological challenge, since oxygen is quite reactive and unstable. High concentrations of oxygen could not be provided for home use because of this instability. Oxygen can, however, be provided in the form of a peroxide and a peroxide decomposition catalyst per US patent publication 2006/0121101 to Ladizinsky. This publication provides such a treatment for intact skin through the use of a dressing that is applied to an area of the skin. The dressing generally has a rupturable reservoir containing an aqueous hydrogen peroxide composition and a hydrogel layer having a peroxide decomposition catalyst. Unfortunately the catalytic decomposition of hydrogen peroxide to oxygen is quite rapid and so the dressing has a layer that is impermeable to oxygen on the outside so that the oxygen is held against the skin for the maximum time possible. While this dressing is useful for small areas of the skin, it should be clear that it is unworkable for large areas or irregularly shaped areas of skin.

Alternatively, Devillez (U.S. Pat. No. 5,736,582) proposes the use of hydrogen peroxide in the place of benzoyl peroxide in skin treatment compositions that also contain solvents for hydrogen peroxide. This allows the hydrogen peroxide to stay below a level that will damage the skin and to stay in solution in greater concentrations.

A solvent such as dimethyl isosorbide along with water is taught as being effective. No peroxide decomposition catalyst is present. Unfortunately, no data on oxygen concentration or generation are given, nor is the time required for oxygen liberation. While this method appears to be an advance over non-oxygen containing compositions, the lack of data makes it difficult to make objective judgments on the overall effectiveness of this approach. Given the concentrations of peroxide, however, it is doubtful that significant volumes of oxygen were generated.

Other proposals for oxygen delivery involve bottles having two compartments; one containing peroxide and the other containing catalyst. The peroxide and catalyst are mixed as they are dispensed from the bottle and oxygen is generated at that time. Such a system, thought effective, can be costly to produce. In addition, the risk remains that the ingredients in the compartments will come in contact with each other due to leakage during transportation or in other ways and the oxygen will be liberated prematurely.

There is a need for an easy-to-use way of applying oxygen to the skin or to a wound. Such a method and/or product should have relatively few components and be intuitive to use, without the need for special dressings, bottles or other awkward requirements. A product that may be used in a manner similar to known products would be most readily accepted by the consumer.

### SUMMARY

The problem discussed above has found a solution to a large degree in the present disclosure, which describes the method of separately encapsulating peroxide and a catalyst that catalyzes the breakdown of peroxide into water and oxygen. The oxygen is released or “liberated” when the peroxide and catalyst come into contact with each other.

The composition produced by the method has microencapsulated peroxide and microencapsulated catalyst that liberate oxygen upon sufficient contact with each. The components may be stored separately or together until use. Upon mixing, oxygen is liberated. The composition can be used for wound healing or for cosmetic applications to deliver oxygen to the skin to help skin elasticity and retard the effects of aging.

To impart additional cosmetically desirable properties, the component compositions may contain other ingredients such as natural or synthetic polymers, moisturizers, humectants, viscosity modifiers, emollients, texture enhancers, UV blocking agents, colorants, pigments, ceramics (fumed silica, titanium dioxide, natural and synthetic clays), antioxidants, fragrances etc.

### BRIEF DESCRIPTION OF THE DRAWINGS

FIG. 1 is a graph of the activity of the four examples or “iterations” of catalase of Formulation A. Activity in U/mg is shown on the Y axis and time in days is shown on the X axis.

FIG. 2 is a graph of oxygen released by microencapsulated catalase of Formulation B in a 0.9% H₂O₂ water solution over time. Dissolved oxygen in ppm is on the Y axis and time from 0 to 1 hour is on the X axis.

FIG. 3 is a graph of dissolved oxygen in ppm released over time for a catalase, hydrogen peroxide microsphere mixture in a ratio of 1:3. Dissolved oxygen is on the Y-axis and time in hours is on the X-axis. A control line (water) is the lower line.

### DETAILED DESCRIPTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

The application of oxygen to the skin can help to alleviate a number of problems brought on by aging such as poor skin health and an excessive presence of visible conditions such as wrinkles, dryness and lower skin elasticity. Oxygen applied to the skin can help to retard these age related effects and improve and maintain skin health. In a similar way, oxygen supplied to a wound can speed healing and reduce scaring. In supplying oxygen to a wound, however, the liberation of the oxygen is desired over a longer period of time than oxygen for application to the skin.

Applying oxygen topically through the application of a liquid or foam composition is a convenient, easy and quick method of delivering the desired benefits discussed above. A two part formulation helps to ensure that the oxygen is available for use and has not been lost during storage, but can present a challenge for product developers because of the need to keep the two ingredients separate and combine them at the proper time.

The liberation of oxygen that is generated “on-demand” may be accomplished by utilizing entrapped chemistries. Examples of such ingredients or chemistries include a catalyst and hydrogen peroxide in a way that no (or virtually no) residue of the ingredients is left behind or absorbed into the skin.

One method of producing microencapsulated materials is taught by Hardy et al. (U.S. Pat. No. 5,496,728) and involves the encapsulation of bleach activator with microorganisms. The method first deodorizes the intact microorganism cells with peroxygen bleach and then contacts the cells with a liquid bleach activator which the cells encapsulate.

WO2006/003581 describes that smaller particles than those known in the art can be produced using a submerged nozzle to which a frequency is applied, preferably in combination with an assistant pressure. Careful shrinkage of the jetted emulsion droplets yielded particles as small as 2 μm. Monodisperse hollow capsules to could be obtained as also described in Bohmer et al. (2006) (Colloids and Surfaces 289, 96-104). in the described system, an assistant pressure not only allows higher jetting rates but also prevents dogging of the nozzle of the device. If no additional pressure is used, polymers such as poly-lactic acid will precipitate at the interface between the fluid to be jetted and the continuous phase.

Another way to arrive al very well-defined particles derived from biodegradable polymers is to use an ink jetting technique in which polymer microparticles are hardened by allowing the microparticles to drop within a liquid (receiving fluid), as taught in U.S. Pat. No. 8,313,676 to Bohmer et al. The receiving fluid is an aqueous solution which can be buffered and can contain additional compounds such as salts, surfactants stabilizers, organic compounds up to 2, 10 or 20%, or other additives. During this initial swelling and/or hardening the receiving fluid is typically not stirred to avoid mechanical damaging, caused for example by collision of the particles with each other. By choosing the appropriate height of the recipient, it can be ensured that the emulsion droplets fall a specific distance by gravity thereby hardening to a certain extent, after which they are removed from the recipient and can be stirred for further hardening. In one embodiment, emulsion droplets ejected from a nozzle are contacted within the receiving fluid with a downwardly inclined surface and start swelling and/or hardening while rolling down or sliding on this surface. Desirably, the inclined surface has a gradually changing slope since it has been found that by allowing the emulsion droplets to roll or slide down a gradually changing slope within the receiving fluid, instead of falling under gravity, they age within the receiving fluid for a specified period of time and monodisperse particles can be obtained with increased uniformity.

Another method of producing microencapsulated materials is that used by Orbis Biosciences (www.orbisbio.com) of Kansas City, Kansas, described in U.S. Pat. No. 6,669,961 to Kim et al. and referred to as precision particle fabrication (PPF) technology. According to the website, PPF involves the use of continuous-flow, high-volume nozzle technology with precise control over key attributes like particle size, composition, coating, and materials. Particles can be produced with a controlled diameter from 2 μm up to 1 mm at and can accommodate almost any active ingredient from small hydrophilic or hydrophobic molecules to macromolecules, polymers, nucleic acids and proteins.

In the method of U.S. Pat. No. 6,669,961, the ingredient to be encapsulated (the core) is surrounded by the material with which it is desired to encapsulate (the shell) and fed through a nozzle. A piezoelectric transducer driven by a wave generator (or other means) is used to vibrate the fluid core/shell stream as it exits the nozzle and break the stream into droplets or particles. This vibration desirably also increases the (downward) velocity of the fluid beyond the velocity produced by the pressure behind the fluid. The nozzle outlet is desirably located below the surface of an aqueous bath, thus avoiding the impact of the particles with the surface of the liquid. The nozzle may also be a dual orifice nozzle with the core exiting an inner nozzle and the shell exiting an outer nozzle surrounding the core.

In the practice of this disclosure, poly(lactic-co-glycolic acid), hereafter referred to as PLGA is desirably used as the shell and hydrogen peroxide and catalase are used (separately) as the core. PLGA is a biodegradable polymer that has been approved by the US Food and Drug Administration (FDA) for in vivo applications.

In order to investigate the effectiveness of the microencapsulated peroxide and microencapsulated catalase, samples of each type were prepared according to the PPF method discussed above. The investigation focused on two release periods for each type of microsphere: (A) A long-acting, 3-day formulation, and (B) a short-acting, 1-hour formulation. The following examples outline the results including successful and unsuccessful approaches.

### Materials Used:

| Chemical | Manufacturer |
|---|---|
| Water, Deionized | In-house source |
| Methylene Chloride, HPLC grade | Fisher Chemical |
| Poly (d,l-lactic-co-glycolic acid) (PLGA), | Lakeshore Biomaterials |
| 50:50 1A | |
| Poly (vinyl alcohol) (PVA), 88% hydrolyzed | Sigma Aldrich |
| Poly (ethylene glycol) (PEG), Mn 300 | Sigma Aldrich |
| Poly (ethylene glycol) (PEG), Mn 2000 | Fisher Chemical |
| Ceresin Wax | Spectrum Chemicals |
| Pluronic F68 (poloxamer 188) | Sigma Aldrich |
| Xanthan Gum, 100% | Now Foods |
| Gelatin, From bovine skin | Sigma Aldrich |
| Catalase, From bovine liver | Sigma Aldrich |
| Hydrogen peroxide (H₂O₂), 30% | Fisher Chemical |

### Formulations

Formulation A—Long-Acting, 72 hour Release (75-100 μm)
Catalase formulation: 4 iterations (PLGA)
Hydrogen peroxide formulation: 2 iterations (Wax, then PLGA)
   Formulation B—Short Acting, 1-hour Release (30-50 μm)
Catalase formulation: 2 iterations (PEG, then PLGA)
Hydrogen peroxide formulation: 1 iteration (PLGA)
   Formulation A: Long-Acting (72 hour) Release: Catalase

Formulation in, and delivery of, proteins from biocompatible polymers (such as PLGA) is a well-characterized approach for extended release applications. The desire was to disperse and encapsulate catalase within 75-100 μm microspheres for a final concentration of 5 wt % catalase in the microspheres, and have the catalase release over a period of 72 hours once exposed to a hydrated environment.

The long-acting catalase formulation went through four iterations, each investigating methods for achieving protein, i.e. catalase, release that was commensurate with the desired 72 hours. The basic process for creating these microspheres was to first create a catalase-rich water phase, emulsify it with an organic-polymer oil phase, and finally make microspheres using PLGA with dichloromethane (methylene chloride) solvent in the PPF process.

The PLGA chosen was a low-molecular weight version (see table above), with an estimated degradation time of 1-2 weeks. To ensure that a considerable amount of catalase was available over 72 hours, each iteration utilized various formulation parameters (catalase loading, water:oil ratios, excipients) to tailor release in a hydrated environment.
Iteration 1: Catalase in water, emulsified at 1:9 water:oil ratio in PLGA
Iteration 2: Catalase in water, emulsified at 1:4 water:oil ratio in PLGA
Iteration 3: Catalase in 50:50 water:PEG 300, emulsified at 1:9 water:oil ratio in PLGA
Iteration 4: Catalase in 50:50 water:PEG 300, emulsified at 1:4 water:oil ratio in PLGA

Following fabrication, the microspheres containing the dispersed catalase were collected, lyophilized into a powder form, and stored at −80 ° C. until needed. A release study was then performed on each formulation to determine the activity per mass of microsphere as a function of time. The microspheres were weighed and placed in microcentrifuge tubes filled with phosphate buffered saline (PBS) for seven days. Samples were taken every 24 hours and measured for activity via bioassay (by CellBiolabs, Inc. of San Diego, Calif., www.cellbiolabs.com).

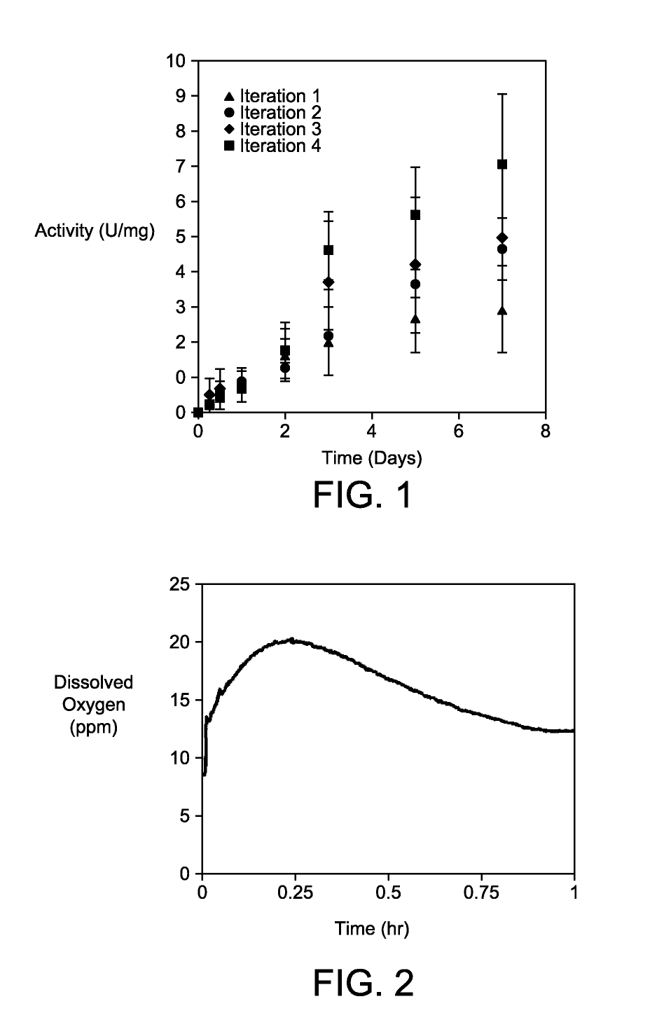

The data demonstrate that the fastest release of catalase occurs when the water phase contains PEG emulsified in a higher volumetric ratio to the PLGA/organic phase (FIG. 1). It's believed this was due to the water-swellable nature of PEG, which allowed faster-forming pore space within the PLGA matrix, and subsequent release of catalase. This was enhanced by having a larger volumetric fraction of aqueous phase to organic phase (1:4 versus 1:9). Iteration 4 therefore had the highest activity.

The above formulation (iteration 4) was tested for in vitro oxygen generation. Catalase microspheres were placed in a closed system in a 0.9% H₂O₂ solution and monitored for 72 hours for dissolved oxygen. Oxygen generation was seen over 72 hours, confirming that catalase was continually released for an extended period.

Formulation A: Long-Acting (72 hour) Release: Hydrogen Peroxide

Microencapsulation of liquids has historically been difficult, especially with liquids that are extremely hydrophilic. Such is the case with hydrogen peroxide. The key issue in creating stable microparticle formulations with disperse aqueous phases, or H₂O₂, is mitigating escape of the aqueous phase prior to use. The encapsulating matrix should desirably be composed of a hydrophobic material, or contain physical or chemical networks that encourage the stable emulsification of the aqueous phase. As a result it was desired to encapsulate concentrated liquid H₂O₂ in 75-100 μm microspheres in a manner that provided a 72-hour release similar to the catalase formulation.

The long-acting H₂O₂ formulation went through two examples or “iterations”, each investigating methods for achieving H₂O₂ entrapment and release that were commensurate with the desired release time of 72 hours. The first iteration used a ceresin wax-based formulation, which was ideal because of the hydrophobic nature of the encapsulating wax material. Similar to the PLGA-solvent-based system as used in the catalase examples above, PPF can also be used in a way that avoids solvents by melting the shell matrix material and allowing it to rapidly cool when exposed to air after exiting the nozzle. The final product is immediately in a dry state. In the case of the first iteration, the aqueous H₂O₂ phase was constantly stirred at high rpm with melted ceresin wax in a 1:19 H₂O₂:wax volume ratio (without PLGA or solvent), and was discharged through a mixing vessel and nozzle, producing droplets of wax.

Upon further investigation, however, it was evident that the entrapment of H₂O₂ in the wax was almost negligible. This was supported by a large fraction of H₂O₂ remaining inside the mixing vessel, a discontinuous product stream during manufacture, and an absence of H₂O₂ release when particles were fractured. Wax for the production of H2O2 particles was therefore considered a failure.

The second iteration moved to a more traditional method of water-oil emulsions, as performed for the catalase particles using PLGA and solvent. The major difference between this iteration and the catalase iterations was that the water phase did not contain catalase, but a concentrated H₂O₂ solution in a 1:9 H₂O₂:oil ratio with 1 wt % poloxamer 188. While the organic-H₂O₂ emulsion was easier to maintain after sonication than a melted wax solution, the final product was not dry, and doing so via lyophilization would sublime the water and H₂O₂. Thus, the final particles were collected and frozen until further use at minus 80° C., which is a temperature at which H₂O₂ is stable.

A release study was then performed to determine the amount of H₂O₂ released per mass of microparticle as a function of time. The wet particles were weighed and placed in microcentrifuge tubes filled with phosphate buffered saline (PBS) for 3 days. Samples were taken every 24 hours and measured via bioassay (by Pierce-Thermo Scientific of Rockford, Ill., www.piercenet.com). Unfortunately, the assay results indicated a likely incompatibility between the assay detection method and the PLGA breakdown byproducts, making results unreliable. The formulation was tested for in vitro oxygen generation. H₂O₂ particles were placed in a closed system in a 0.3% catalase in water solution and monitored for 2 hours for dissolved oxygen.

Despite the apparent initial entrapment of H₂O₂ microbubbles within the PLGA matrix, the retention post-processing was poor. Dissolved oxygen concentrations demonstrated a flat-line profile (with some intermittent equipment noise), warranting an improvement in formulation.

Formulation B: Short-Acting (1 hour) Release: Catalase

With the knowledge gained during the Formulation A attempts, an interest in producing smaller particles for a lotion-based application was desired. The desire was that the particles be less than 50 μm, preferably 30 μm to make catalase (and subsequent oxygen generation) available within 1 hour after being spread on a surface.

To ensure fast availability of catalase, the first iteration attempted to use a low molecular weight (300 Mn) PEG. The PEG was solid at room temperature, and would deform when spread on a surface. To manufacture particles, however, PEG in its melt form would be needed, which typically comes with a monodisperse size limitation of 100 μm or higher. Moving lower in size would create a broad size distribution.

To manufacture the particles, a concentrated catalase solution was emulsified with melted PEG, such that the overall weight fraction of catalase was 1%. The solution was then frozen and lyophilized to create a finely-dispersed catalase phase with no aqueous component. This solid was then re-melted and sprayed through a nozzle using PPF as attempted with the H₂O₂/Wax setup in Formulation A without PLGA or solvent. The ability to make PEG-catalase particles continuously, however, became increasingly difficult as under heated conditions, catalase aggregated and caused nozzle clogging. Particles that were made successfully were not completely cooled, and exhibited heterogeneous distribution of the catalase. PEG for the production of catalase particles was therefore considered a failure.

The second catalase iteration returned to using PLGA and solvent as the shell, which was successful in Formulation A. Due to the large decrease in particle size desired, it was believed that the release rate of catalase would be increased compared to the previous 72 hour formulation. In addition, a water-swellable component, gelatin, was included in the concentrated catalase phase at 0.5 w/v %, to expedite the release rate, i.e. 5 mg/mL gelatin in water was used with 100 mg/mL catalase, emulsified at a 1:9 water:oil ratio and sprayed through a nozzle using PLGA and dichloromethane. Following fabrication, the particles were subjected to the same release study and assay as performed previously (i.e. in a 0.9% H₂O₂ water solution), and demonstrated an activity at least 10 times that of the first formulation at the 2 hour time in a lotion, as opposed to water only (FIG. 2).

The second catalase iteration formulation was tested for real-time oxygen generation testing, which also showed similar trends and magnitudes of dissolved oxygen as seen in the first formulation, only on a shorter time scale.

Formulation B: Short-Acting (1 hour) Release: Hydrogen Peroxide

The Formulation B H2O2 iteration used 3% peroxide gelled by adding 0.1 w/v % xanthan gum and 0.5 w/v % gelatin. The gel phase was then emulsified at a 1:9 water:oil ratio and sprayed through a nozzle with the shell PLGA/dichloromethane. The particles were frozen at −80 C to maintain stability after fabrication.

Tests were performed on Formulation B catalase (second iteration) and Formulation B H₂O₂ particles separately, in dilute solutions of the complementary components. Specifically, catalase particles were evaluated in a 0.9% H₂O₂ solution, and H₂O₂ particles were evaluated in a 0.3% catalase solution. Results indicated that each particle type is able to generate oxygen in controlled environments as desired, and the oxygen concentration is similar between the two formulations.

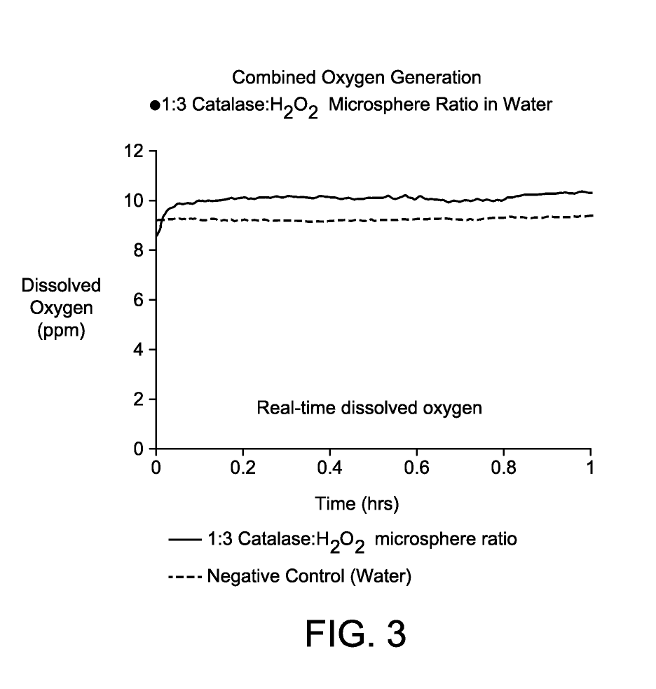

Following separate testing, the Formulation B particles were combined in a 1:3 catalase (second iteration):peroxide ratio in a closed water system and monitored for dissolved oxygen. The levels were compared against a negative control group (water). The results indicated that these particles, when tested together in water, are able to generate ongoing oxygen profiles similar to when tested individually, as seen graphically in FIG. 3.

While the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

What is claimed is:

1. A composition for the delivery of oxygen comprising microencapsulated peroxide and microencapsulated catalyst that liberate oxygen upon contact with each other.

2. The composition of claim 1 wherein said peroxide and/or catalyst is encapsulated with PLGA in the presence of a solvent upon spraying through a nozzle.

3. The composition of claim 2 wherein said microencapsulated peroxide and/or encapsulated catalyst have a size between 2 microns and 1 mm.

4. The composition of claim 3 wherein said microencapsulated peroxide and/or encapsulated catalyst have a size between 50 and 100 microns.

5. The composition of claim 1 wherein said microencapsulated peroxide and microencapsulated catalyst are stored separately until use.

6. The composition of claim 1 wherein said microencapsulated peroxide and microencapsulated catalyst are stored together until use.

7. The composition of claim 1 wherein said peroxide comprises hydrogen peroxide.

8. The composition of claim 1 wherein said catalyst is selected from the group consisting of catalase, manganese dioxide and a base.

9. The composition of claim 1 further comprising natural or synthetic polymers, moisturizers, humectants, viscosity modifiers, emollients, texture enhancers, UV blocking agents, colorants, pigments, ceramics (fumed silica, titanium dioxide, natural and synthetic clays), antioxidants, fragrances and combinations thereof.
